(19) United States
(12) Patent Application Publication
(10) Pub. No.: US 2009/0036931 A1
(43) Pub. Date: Feb. 5, 2009

(54) FOOT SURGERY BONE PLATE, AND SYSTEM COMPRISING BONE PLATE AND INSERTION AID

(75) Inventors: Uwe Pech, Tuttlingen (DE); Roland Worner, Tuttlingen-Mohringen (DE)

Correspondence Address: BACHMAN & LAPOINTE, P.C., 900 CHAPEL STREET, SUITE 1201, NEW HAVEN, CT, 06510, US

(73) Assignee: NORMED MEDIZIN-TECHNIK VERTRIEBS-GMBH, Tuttlingen (DE)

(21) Appl. No.: 12/177,944

(22) Filed: Jul. 23, 2008

(30) Foreign Application Priority Data
Aug. 4, 2007 (DE) 10 2007 036 943.5

(51) Int. Cl. A61B 17/70; A61B 17/56; A61F 5/00

(52) U.S. Cl. 606/280; 606/62; 606/86 B

(57) ABSTRACT

A foot surgery bone plate for osteosynthesis and/or fixation of foot-bone segments, with a plate-shaped bearing portion which has at least one through-opening extending from a top face of the bone plate to a bottom face of the bone plate and provided for a bone screw, which bearing portion is designed for external fixation on a first bone segment. An intramedullary portion is provided which has at least one through-opening extending from the top face of the bone plate to the bottom face of the bone plate and provided for a bone screw, which intramedullary portion is designed for introduction into a second bone segment. A system is provided comprising a bone plate and an insertion aid.

### BACKGROUND OF THE INVENTION

The invention relates to a foot surgery bone plate for osteosynthesis and/or fixation of foot-bone segments.

In osteosynthesis of foot bones, for example for treating hallux valgus or hallux rigidus or for performing Lapidus arthrodesis procedures, bone plates are used which, with the aid of bone screws, are secured from the outside on two bone segments that are to be fixed to each other. The force transmission between the bone segments takes place via the bone screws passing through the bone plate. Depending on the arrangement of the bone screws and on the stability of the bone substance, the maximum transmissible force, and therefore the stability of the implant system, varies considerably from patient to patient. In addition, it is in most cases difficult to achieve a defined arrangement of the bone plates externally on the bone segments that are to be fixed. Implant failures often occur, which adversely affect the chances of healing and which make a repeat surgical intervention necessary.

The object of the invention is to propose an alternative foot surgery bone plate that can be used for displacement osteotomies and that ensures a high degree of stability of the connection between two bone segments. The object is also to propose a system that comprises such a bone plate and that permits simple and defined securing of the bone plate on two bone segments. This object is achieved, in respect of the foot surgery bone plate, by the features of claim 1 and, in respect of the system, by the features of claim 10. Advantageous developments of the invention are set forth in the dependent claims. The scope of the invention also covers all combinations of at least two of the features disclosed in the description, claims and/or figures.

### SUMMARY OF THE INVENTION

The invention is based on the concept of designing the foot surgery bone plate in such a way that it no longer has to be secured by bone screws on the outside of both foot-bone segments. Instead, the foot surgery bone plate designed according to the concept of the invention comprises, in addition to a bearing portion for external fixation on a first, in particular distal bone segment, also an intramedullary portion by means of which it can be secured inside a medullary cavity of a second of the two bone segments to be connected to each other. The second bone segment inside which the intramedullary portion is received is preferably a proximal bone segment. In order to fix the position of the intramedullary portion inside the second bone segment, the intramedullary portion has at least one through-opening, which extends from its top face to its bottom face and through which a bone screw can be guided. The intramedullary portion is preferably inserted into the medullary cavity of the associated bone segment by means of the foot surgery bone plate being driven into an end face of the second bone segment, and by subsequent screwing of the intramedullary portion to the second bone segment using at least one bone screw. After the intramedullary portion has been secured in the inside of the associated bone segment, the foot surgery bone plate can be placed with its plate-shaped bearing portion on the outer face of the first bone segment and can be fixed to the latter by screwing. The provision of the intramedullary portion means that the foot surgery bone plate, and the connection between two foot-bone segments, has a high degree of stability against twisting and shifting. Moreover, the introduction of force into the second bone segment with the intramedullary portion located therein takes place uniformly, as a result of which the likelihood of an implant failure is minimized and more rapid and successful healing is ensured. Moreover, by providing a foot surgery bone plate designed according to the concept of the invention, it is possible to place weight again on the operated foot immediately after the operation. Furthermore, because of the stable connection of the bone segments, there is no need for complete bone contact following the bone incision, as a result of which it is possible to perform open-wedge incisions and also displacement incisions. The foot surgery bone plate designed according to the concept of the invention is suitable in particular for the treatment of all types of hallux valgus and hallux rigidus by displacement and fixing of two foot-bone segments relative to each other. It is also possible, particularly with a suitably long design of the intramedullary portion, to stiffen the tarsometatarsal joint (Lapidus arthrodesis).

In a development of the invention, provision is advantageously made for the intramedullary portion to be pointed at its free axial end, in order to make it easier to insert (drive) the intramedullary portion into the medullary cavity of the second foot-bone segment.

An embodiment is preferred in which the intramedullary portion is not rotationally symmetrical, so as to avoid a rotation within the bone segment and thus ensure a stable position. The intramedullary portion is preferably designed as a flat portion with a top face extending transverse to the longitudinal extent, and with a bottom face extending approximately parallel to the top face. The tip or point is advantageously formed in such a way that the top face and bottom face run toward each other in the area of the free end of the intramedullary portion, i.e. each form oblique planes that extend at an angle to a longitudinal center axis. The transverse extent of the intramedullary portion also preferably narrows in the area of its free end directed away from the bearing portion.

Of particular advantage is an embodiment in which the intramedullary portion has several through-openings spaced apart from one another in the longitudinal direction of the intramedullary portion and provided in each case for one bone screw. The intramedullary portion is preferably fixed in the second bone segment by means of at least two bone screws spaced apart from each other and extending through the intramedullary portion. The through-openings are preferably located on a longitudinal center axis of the intramedullary portion.

An embodiment is particularly preferred in which the bottom face of the foot surgery bone plate has a concave curvature transverse to its longitudinal extent, at least in its bearing portion, in order to ensure optimal contact on a large surface area of the outer face of the bone segment. An embodiment is particularly preferred in which not only the bearing portion, but also the intramedullary portion, has a concave curvature in the transverse direction on its bottom face directed toward the bone. Viewed in the longitudinal direction, the bottom face of the bearing portion preferably merges steplessly into the bottom face of the intramedullary portion, which bottom face is preferably also concavely curved in the transverse direction. The concave curvature of the intramedullary portion further increases the positional stability inside the bone segment.

In a development of the invention, provision is advantageously made for the bearing portion, seen from its top face to its bottom face, to be thicker than the intramedullary portion. The bearing portion is advantageously about 0.5 mm thicker than the intramedullary portion. This difference in thickness is preferably afforded, on the top face, by an oblique and in particular stepless transition between the bearing portion and the intramedullary portion.

To allow the bearing portion to bear across the greatest possible surface area on the outer face of the in particular distal first bone segment, an advantageous embodiment is one in which, transverse to the longitudinal extent of the bone plate, the bearing portion is wider than the intramedullary portion. The bearing portion preferably extends in fact to a transverse axis running perpendicular to the longitudinal center axis.

To permit optimal fixation of the bearing portion on the first bone segment assigned to it, an advantageous embodiment is one in which three through-holes are arranged in the bearing portion, of which one of the through-holes is preferably arranged on the imaginary continuation of the longitudinal center axis of the intramedullary portion, that is to say in a line with the through-openings of the intramedullary portion. In a plan view of the bone plate, the other two through-openings are preferably offset to the right and left of and in the axial direction from the central through-opening and thus allow introduction of bone screws in different bone segment areas.

A particularly good hold of the bearing portion on the associated bone segment can be obtained by the longitudinal center axes of the outer through-openings forming between them an in particular acute angle, preferably of between approximately 10° and 20°.

To make it easier to drive the intramedullary portion in the longitudinal direction into the medullary cavity of the second bone segment, an advantageous embodiment is one in which the intramedullary portion of the foot surgery bone plate has a central and at least approximately flat portion that extends in the longitudinal direction and that comprises the through-openings spaced apart in the longitudinal direction. Arranged to the side of the central, flat portion, there are advantageously respective flank portions that also extend in the longitudinal direction, the thickness of the flank portions advantageously decreasing toward the outside transverse to the longitudinal extent, i.e. the flank portions are designed falling away toward the outside.

The invention is also directed at a system comprising a foot surgery bone plate, as described above, and an insertion aid for more easily driving the intramedullary portion of the foot surgery bone plate into a bone segment, preferably in the longitudinal direction of the bone segment. The insertion aid is preferably distinguished by the fact that the foot surgery bone plate can be secured releasably thereon, particularly with its bearing portion. For example, the foot surgery bone plate can be secured thereon by screwing. For this purpose, at least one of the through-openings in the bearing portion is preferably provided with an inner thread.

For this purpose, a recess that is at least partially congruent to the bearing portion is preferably provided on a bottom face of the insertion aid, and a strike face of the insertion aid, that is to say a face that can be struck with a hammer instrument, advantageously extends perpendicular to the bottom face of the insertion aid.

Of particular advantage is an embodiment of the insertion aid in which it serves not only as an insertion aid but at the same time supports a drill jig or is designed as such. The drill jig is advantageously oriented along the longitudinal extent of the intramedullary portion and permits defined drilling of holes from outside into the second bone segment with the intramedullary portion received therein.

To allow this, the drill jig extends at least approximately parallel to the bone plate secured on the insertion aid, and it has several through-holes arranged in alignment with the through-openings of the intramedullary portion. The operating surgeon can guide his drill through these through-holes and thus exactly position the drilled holes in such a way that said drilled holes coincide with the through-openings of the intramedullary portion and if appropriate even pass through these.

It is preferable that a screwdriver is not passed directly through a through-hole, and instead a protective sleeve can be received in the through-hole and prevents damage to the drill jig or to the through-hole when the screws are being screwed in. To be able to precisely guide the drill when forming a drilled hole, and to avoid damage to the protective sleeve during the drilling procedure, an embodiment is of advantage in which a drill sleeve can be received in the protective sleeve, the drill sleeve having a smaller free internal diameter than the protective sleeve. Alternatively, it is conceivable to design the drill sleeve to be received directly in a through-hole.

It is within the scope of this development of the invention to provide in each through-hole a protective sleeve together with a drill sleeve, or only a protective sleeve and/or only a drill sleeve.

### BRIEF DESCRIPTION OF THE DRAWINGS

Other advantages, features and details of the invention will become apparent from the following description of preferred illustrative embodiments and by reference to the drawings. These drawings indicate dimensions of preferred illustrative embodiments. The dimensions, and dimension ratios arising from them, are disclosed as being essential to the invention and claimed as such. However, the dimensions and dimension ratios in the drawings are not intended to restrict the scope of protection of the invention.

In the drawings:

FIG. 1a shows a perspective view of a foot surgery bone plate from below,

FIG. 1b shows a plan view, enlarged on a 2:1 scale, of the foot surgery bone plate according to FIG. 1a,

FIG. 1c shows a longitudinal cross-sectional view of the foot surgery bone plate along the section line A-A according to FIG. 1b,

FIG. 1d shows a cross-sectional view of the foot surgery bone plate along the section line B-B according to FIG. 1b,

FIG. 1e shows a view of the foot surgery bone plate from behind, looking toward a plate-shaped bearing portion,

FIG. 2a shows a perspective view of an alternative foot surgery bone plate,

FIG. 2b shows preferred dimensions of the foot surgery bone plate, seen in a view from above,

FIG. 2c shows a longitudinal cross-sectional view of the foot surgery bone plate along the section line A-A according to FIG. 2b,

FIG. 2d shows a sectional view of the foot surgery bone plate along the section line B-B according to FIG. 2b,

FIG. 2e shows a view of the foot surgery bone plate from behind, looking toward a plate-shaped bearing portion,

FIG. 3a shows a plan view of an insertion aid for a foot surgery bone plate,

FIG. 3b shows a view of the insertion aid according to FIG. 3a from behind, looking toward its strike face,

FIG. 3c shows a view of the insertion aid from below,

FIG. 3d shows a cross-sectional view of the insertion aid along the section line B-B according to FIG. 3a,

FIG. 3e shows a longitudinal cross-sectional view of the insertion aid along the section line A-A according to FIG. 3a,

FIG. 4 shows a perspective view of a system comprising foot surgery bone plate and insertion aid,

FIG. 5 shows a perspective view of the insertion aid obliquely from below,

FIG. 6 shows a perspective view of a system comprising foot surgery bone plate and insertion aid, seen obliquely from above, and

FIG. 7a to FIG. 7k show individual steps in a hallux valgus operation using a system comprising an insertion aid and a bone plate with bearing portion and intramedullary portion.

### DETAILED DESCRIPTION

In the figures, identical parts, and parts having an identical function, are designated by the same reference signs.

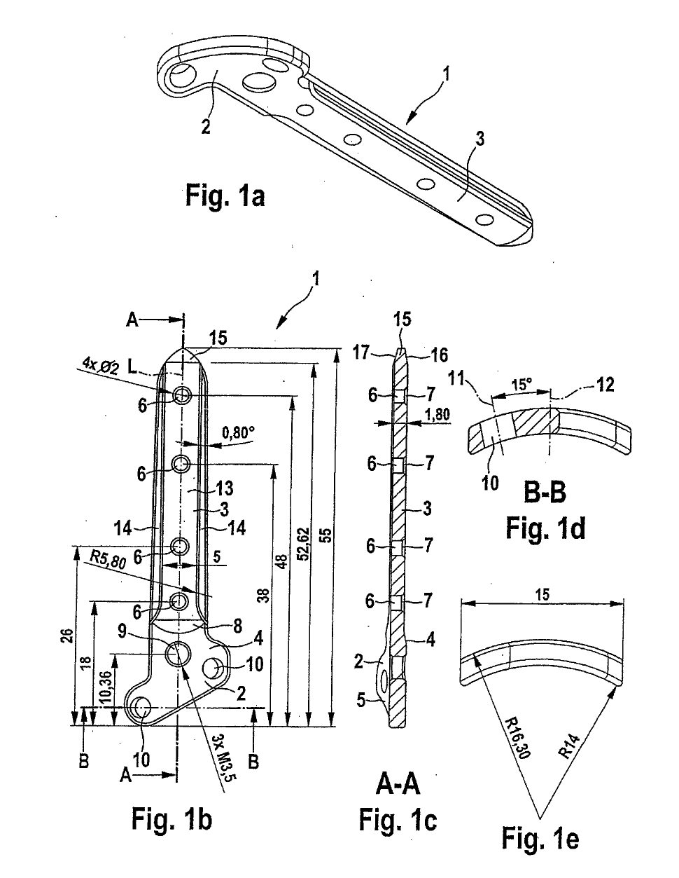

FIGS. 1a to 1e show different views of a first preferred illustrative embodiment of a foot surgery bone plate 1 (hereinafter bone plate 1) that is made of a titanium alloy and can be used in displacement osteotomies. The bone plate 1 is divided into two integrally connected portions, namely a plate-shaped bearing portion 2 to bear externally on and be fixed to a first, in particular distal foot-bone segment, and, adjoining the bearing portion 2, an intramedullary portion 3 to be received in a second, in particular proximal bone segment.

The bone plate 1 has a top face 4 and, extending approximately parallel to the latter, a bottom face 5. As will be seen in particular from FIGS. 1a, 1d and 1e, the bottom face 5 has a continuous concave curvature transverse to the longitudinal extent of the bone plate 1. As will be seen in particular from FIG. 1e, the radius of curvature R of the bottom face 5, curved concavely in the transverse direction, is 14 mm. It will be noted that, on the bottom face, there is a smooth stepless transition, with no height difference, between the intramedullary portion 3 and the bearing portion 2. As will also be seen from the figures, the bearing portion 2 has a greater width in the transverse direction, i.e. transverse to the longitudinal extent of the bone plate 1, than does the intramedullary portion 3. The bearing portion 2 has an asymmetrical configuration and extends at an angle, i.e. obliquely with respect to an imaginary transverse axis of the bone plate 1. The outer contour of the bearing portion 2 is rounded.

Along a longitudinal center axis L of the bone plate 1, a total of four through-openings 6 for bone screws (not shown) are provided in the intramedullary portion 3. As will be seen in particular from FIG. 1c, the through-openings 6 with an internal diameter of 2 mm are provided, at their end located on the top face 4, with a beveled edge 7 for easier locating.

In the bearing portion 2, which merges into the intramedullary portion 3 via an obliquely extending transition 8, a central through-opening 9 is provided on the longitudinal center axis L and receives a bone screw (not shown) for fixing the bearing portion 2 externally on the first bone segment. The internal diameter of the central through-opening 9 is 3 mm and is provided with a metric inner thread having an inner thread diameter of 3.5 mm. A fixing screw, of an insertion aid to be explained in detail below, can be screwed into the inner thread of the through-opening 9, such that the bone plate 1 can be secured with its bearing portion 2 releasably thereon. In addition to the through-opening 9, other through-openings 10 with an internal diameter of 3 mm are provided, in the plane of the drawing (cf. FIG. 1b), to the right and to the left of the through-opening 9. The through-openings 9, 10 are arranged offset relative to one another in the axial direction.

As will be seen from FIG. 1d, the longitudinal center axis 11 of the left-hand through-opening 10 in the plane of the drawing forms an acute angle of approximately 15° with a longitudinal center axis 12 of the central through-opening 9. The same applies to the longitudinal center axis (not shown) of the right-hand through-opening 10 in the plane of the drawing according to FIG. 1b and the longitudinal center axis 12 of the central through-opening 9, which likewise enclose an acute angle of 15° to each other.

As will be seen from FIG. 1d, the material thickness in the intramedullary portion 3 and in a central flat portion 13 is smaller than in the bearing portion 2. The material thickness is 1.8 mm in the intramedullary portion and 2.3 mm in the bearing portion 2.

On its top face 4, the intramedullary portion 3 of the bone plate 1 has the central flat portion 13, which extends in the longitudinal direction and is contoured approximately as a rectangle and at the lengthwise sides of which there extends in each case a flank portion 14. The flank portions 14 fall away toward the outside in the transverse direction, in other words narrow in the transverse direction.

The intramedullary portion 3 is provided with a tip 15 and, as can be seen from FIG. 1c, said tip 15 is formed by two surface portions 16, 7 that extend obliquely forward and are formed respectively by the top face 4 and bottom face 5 of the intramedullary portion 3. Moreover, the intramedullary portion 3 also narrows, in the transverse direction, symmetrically toward its free end, such that the tip 15 as a whole acquires a shallow configuration.

The tip 15 allows the bone plate 1 to be driven with its intramedullary portion 3 in the longitudinal direction into a medullary cavity of a second foot-bone segment, in particular from the direction of the front face thereof.

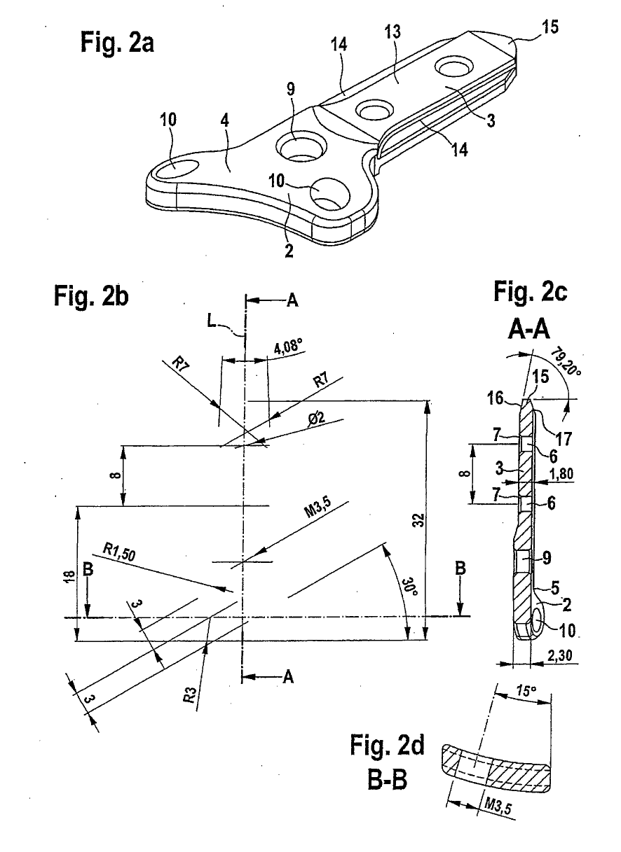

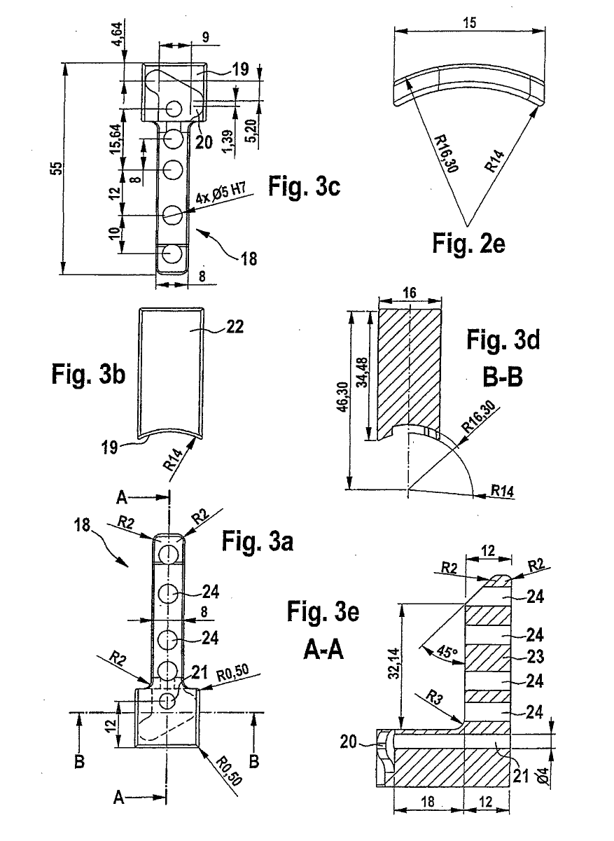

FIGS. 2a to 2e show a bone plate 1 with an alternative configuration. For the sake of clarity, the bone plate is not indicated in FIG. 2b. The situation in respect of the indicated dimensions corresponds to the situation of the bone plate 1 according to FIG. 1b.

The main difference between the bone plate 1 according to FIGS. 2a to 2e and the bone plate shown in FIGS. 1a to 1e is essentially the shorter design of the intramedullary portion 3. In the illustrative embodiment shown, the intramedullary portion 3 has only two through-openings 6 spaced apart from each other in the longitudinal direction and lying on the longitudinal center axis L, each of them with a beveled edge 7 at the top face. The shape of the bearing portion 2 corresponds to the design of the bearing portion 2 according to FIGS. 1a to 1e, including the arrangement and design of the three through-openings 9, 10.

As will be seen in particular from FIG. 2c, the material thickness in the bearing portion 2 is 0.5 mm greater than in the intramedullary portion 3. The figure also shows the angle of 79.2° between the upper surface portion 16, formed by the top face 4, and the longitudinal center axis L.

The bone plate 1 shown in FIGS. 2a to 2e can be used in particular to perform hallux valgus and hallux rigidus osteotomies.

FIGS. 3a to 3e show different views of an insertion aid 18 for driving the bone plate 1 in its intramedullary portion 3 into the medullary cavity of a second bone segment, in particular a proximal second bone segment, of a foot.

On its bottom face 19, the insertion aid 18 has a recess 20 which has a shape congruent to the bearing portion 2 and into which the bearing portion 2 can be placed. A fixing screw can be passed through a channel 21 and screwed into the inner thread of the central through-opening 9 in the bearing portion 2, such that the bone plate 1 can be secured releasably on the insertion aid 18, or on the bottom face 19 thereof. The figure shows the concave curvature of the bottom face 19 in the transverse direction. The radius of curvature corresponds here to the radius of curvature of the bottom face of the bone plate 1.

Extending at right angles to the bottom face 19 there is a rear strike face 22 against which a hammer instrument can be struck in order to drive the bone plate 1 into the medullary cavity of the second foot-bone segment.

A drill jig 23 of the insertion aid 18 extends parallel to the secured bone plate 1. Four through-holes 24 spaced apart in the longitudinal direction are arranged in the drill jig 28, the longitudinal center axes of these through-holes 24 being in alignment with the longitudinal center axes of the through-openings 6 in the intramedullary portion 3, such that, after the intramedullary portion 3 has been driven into the second bone segment, it is possible to drill via the through-holes 24 from the outside into the second bone segment, the alignment of the through-holes 24 with the through-openings 6 ensuring that the drilled holes coincide with the through-openings 6, which in turn ensures an exact arrangement of bone screws for fixing the intramedullary portion 3 in the second bone segment.

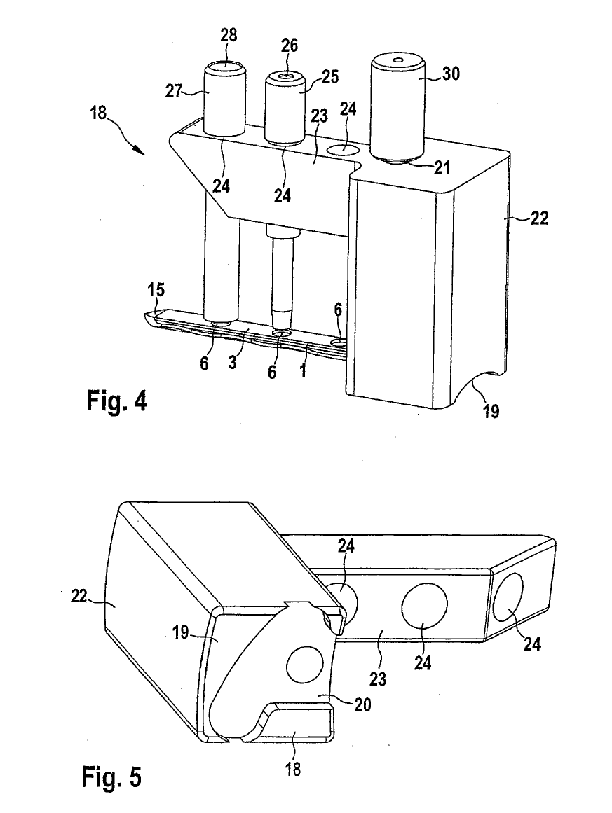

FIG. 4 shows a perspective view of the insertion aid 18, or of a system composed of insertion aid 18 and bone plate 1. The figure shows the rear strike face 22 and the concavely curved bottom face 19 on which the bone plate 1 is secured. It can also be seen that the intramedullary portion 3 extends parallel to the drill jig 23, the through-holes 24 being in alignment with the through-openings 6 in the intramedullary portion 3.

To permit drilling into the second bone segment in which the intramedullary portion 3 is received after being driven in, a drill sleeve 25 is provided in whose axial channel 26 a drill is guided exactly in the longitudinal direction. The drill sleeve 25 can be fitted into each desired through-hole 24 and is precisely oriented by the latter with respect to the associated through-opening 6 in the intramedullary portion. After a hole has been drilled into the second bone segment, the drill sleeve 25 is removed and is replaced by a protective sleeve 27 whose axial channel 28 has a larger internal diameter than the axial channel 26 of the drill sleeve 25, so as to be able to receive a screwdriver for tightening the bone screw (not shown). In an alternative embodiment, the drill sleeve 25 is dimensioned in such a way that it can be received in a protective sleeve 27, such that the protective sleeve 27 is first fitted into a through-hole 24 and, thereafter, the drill sleeve 25 is fitted onto the protective sleeve 27 or inserted into it. After a hole has been drilled, the drill sleeve 25 can then be removed from the protective sleeve 27, and a corresponding bone screw can be screwed into the drilled hole in such a way that the bone screw passes through a through-opening 6 of the intramedullary portion 3 transverse to this longitudinal extent.

As will be seen from FIG. 4, a fixing screw 30 is received in the channel 21 extending from the top face to the bottom face of a main body of the insertion aid 18, the lower outer thread of the fixing screw 30 cooperating with the inner thread of the central through-opening 9 in the bearing portion 2 of the bone plate 1.

FIG. 5 shows an insertion aid 18 in a view from below. The figure indicates the congruently shaped recess 20 for the bearing portion 2 of the bone plate 1 on the bottom face 19, and the longitudinally extending drill jig 23 with its through-holes 24 for receiving protective sleeves and/or drill sleeves 25, 27.

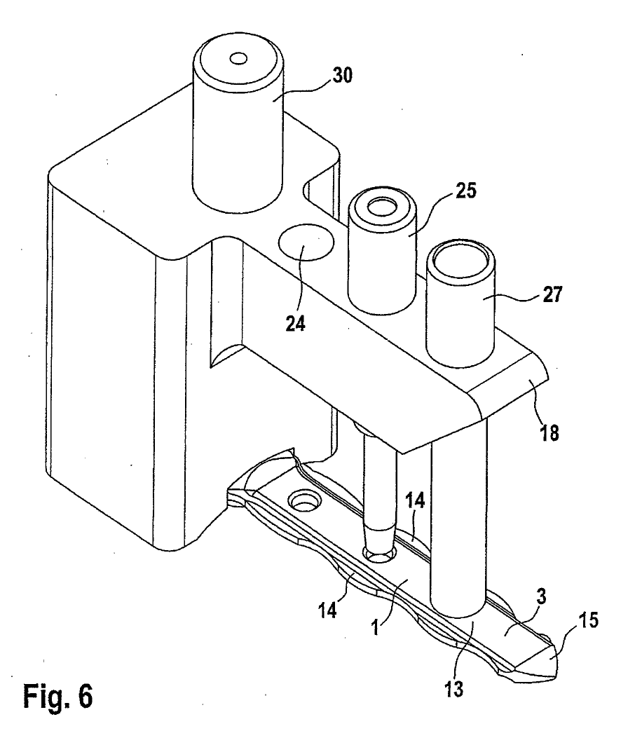

FIG. 6 shows another perspective view of the insertion aid 18, or of the system composed of insertion aid 18 and bone plate 1. Compared to the bone plates 1 shown in FIGS. 1a to 1e and FIGS. 2a to 2e, the edge of the flank portions 14 of the bone plate 1 according to FIGS. 4 and 6 has an undulated configuration in the longitudinal direction.

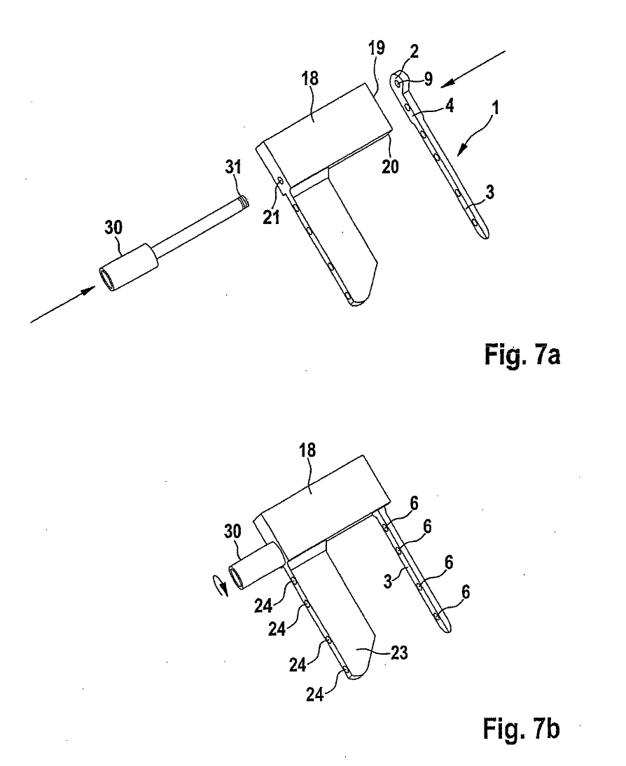

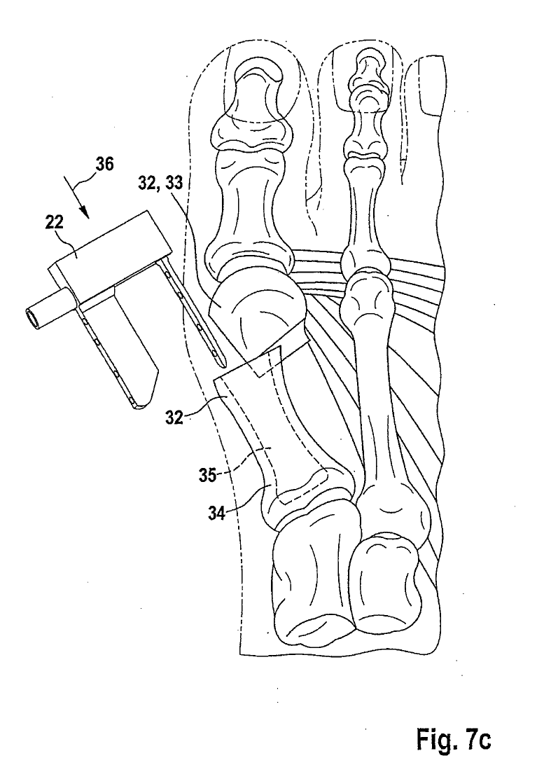

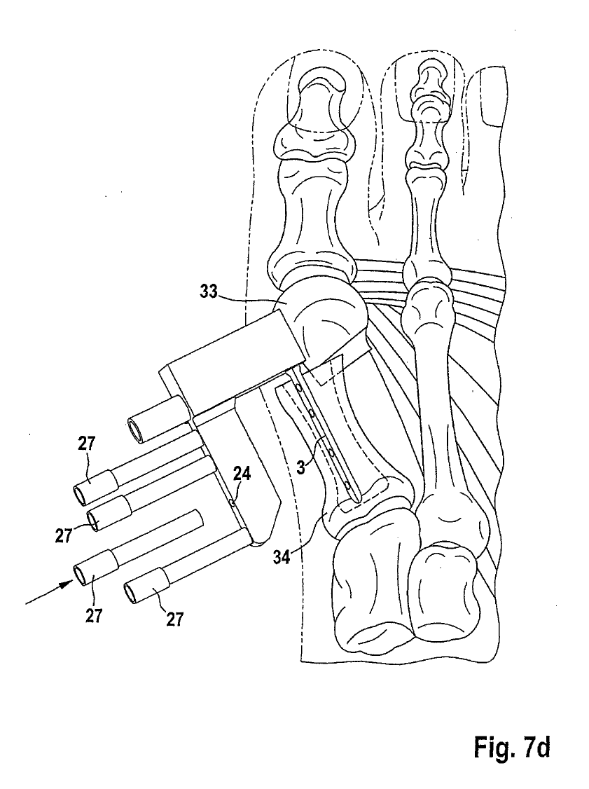

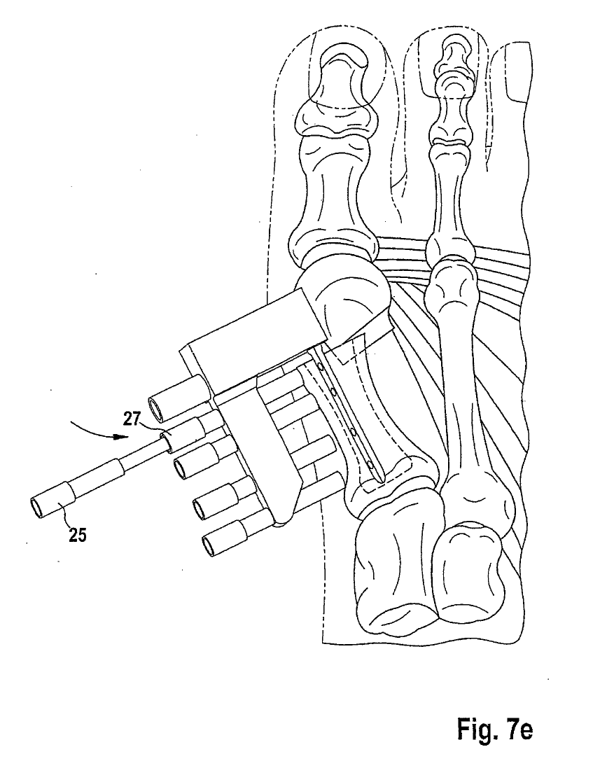

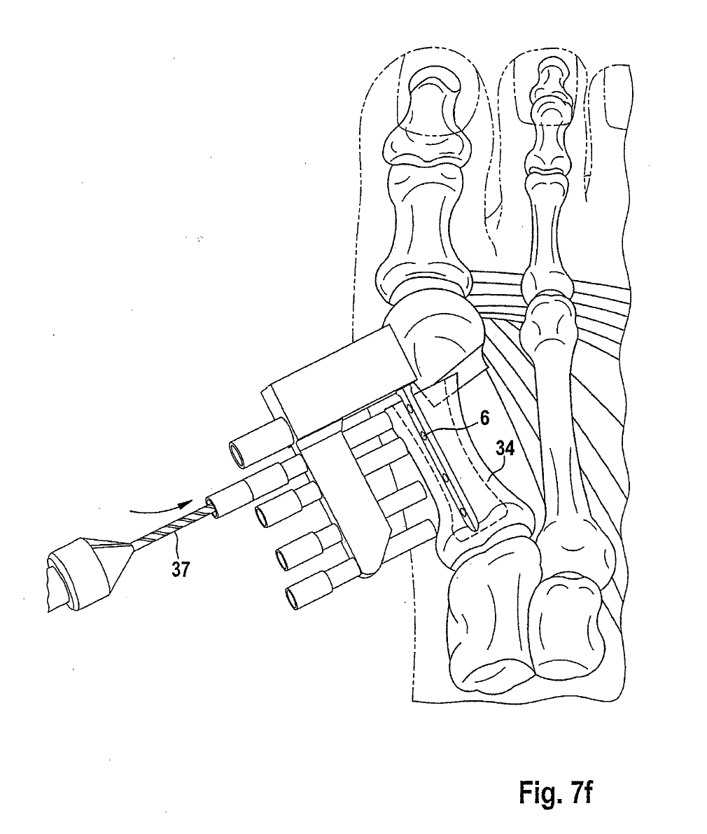

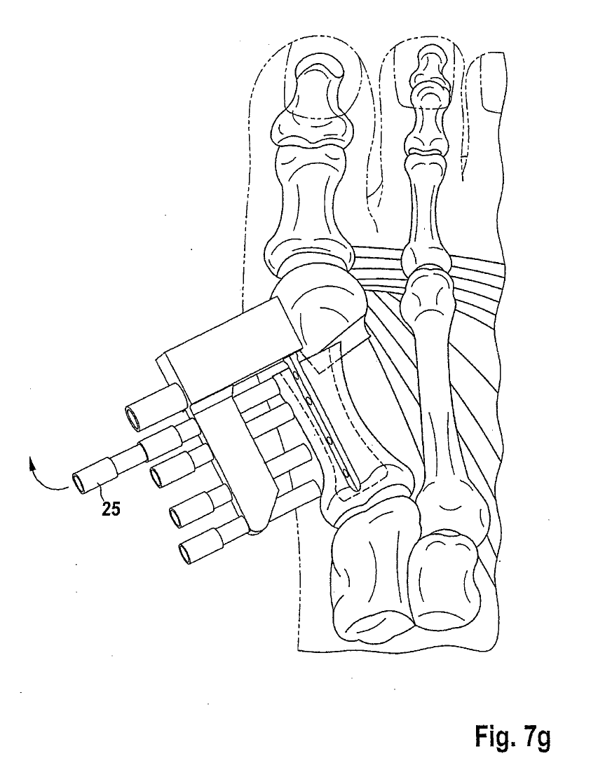

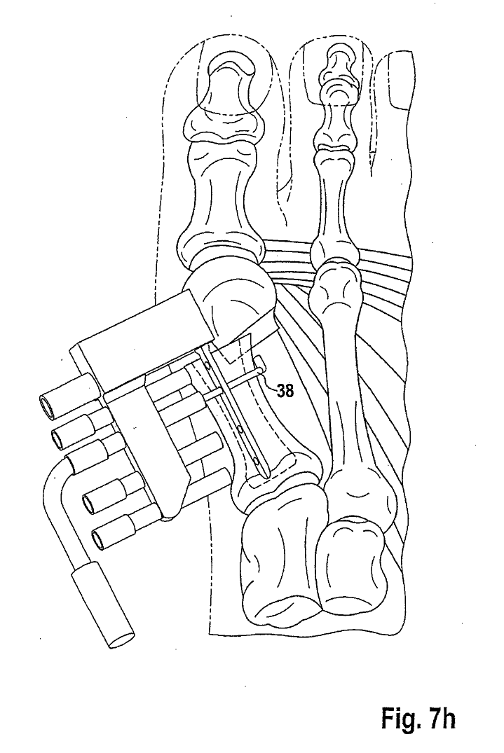

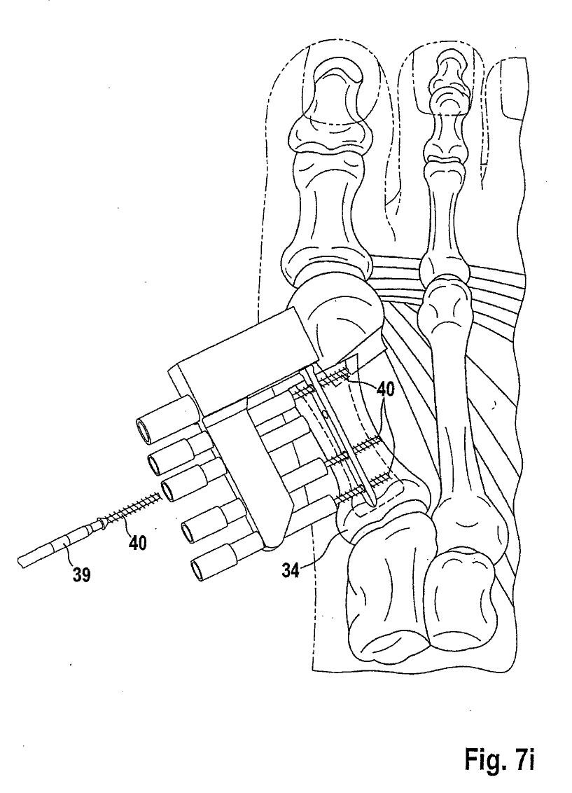

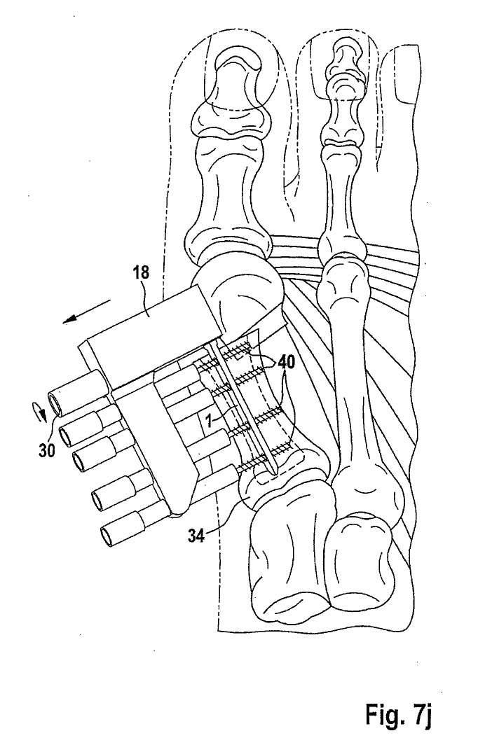

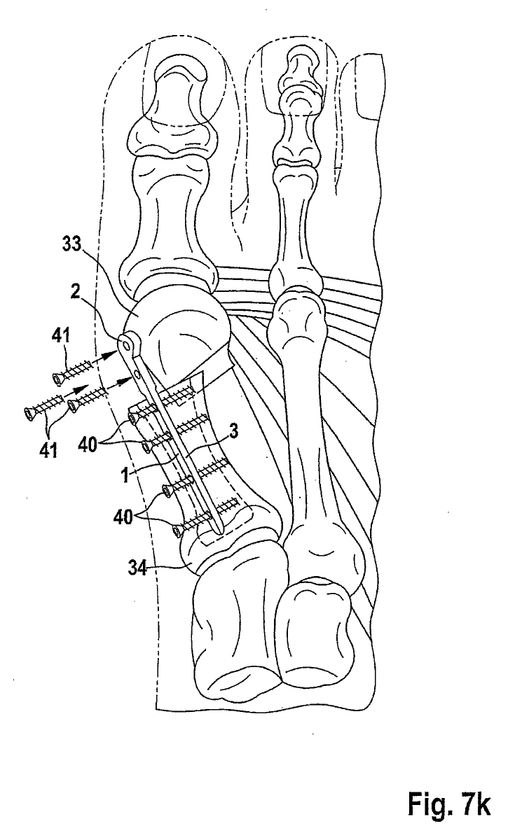

A surgical technique for a hallux valgus operation using a system composed of insertion aid 18 and bone plate 1 will now be described with reference to FIGS. 7a to 7k.

Before or after dividing a foot bone 32 into two bone segments 33, 34 and displacing the distal first bone segment 33 in the plane of the drawing toward the right in the direction of the center of the foot, the bone plate 1 is placed with its bearing portion 2 into the recess 20 on the bottom face 19 of the insertion aid 18, specifically in such a way that the top face 4 of the bone plate 1 is oriented in the direction of the insertion aid 18 (see FIG. 7a). Thereafter, the fixing screw 30 is introduced into the channel 21, as can be seen from FIG. 7b, such that its outer thread 31 provided at the lower end meshes with the inner thread of the central through-opening 9 in the bearing portion 2, with the result that the bone plate 1 is fixed on the insertion aid 18 in such a way that the intramedullary portion 3 extends parallel to the drill jig 23 of the insertion aid 18, and the through-holes 24 in the drill jig 23 are in alignment with the through-openings 6 in the intramedullary portion 3.

After the foot bone 32 has been divided into the first, distal bone segment 33 and the proximal, second bone segment 34, the intramedullary portion 3 is driven into the medullary cavity 35 of the second bone segment, from the direction of the front end thereof, using the insertion aid 18, this driving-in movement being effected by impacting the strike face 22 with a hammer instrument. The driving-in movement is symbolized by the arrow 36 (see FIG. 7c).

FIG. 7d shows the bone plate 1 with the intramedullary portion 3 already driven into the second bone segment. The figure shows protective sleeves 27 that can be received in the through-holes 24. The operation can be performed using just one protective sleeve 27, which can be inserted as necessary into the desired through-hole 24.

As will be seen from FIG. 7e, a drill sleeve 25 can be received in each protective sleeve 27, the internal diameter of the drill sleeve 25 corresponding to the external diameter of a drill plus a minimum clearance. The external diameter of the drill sleeve 25 corresponds in a lower portion to the internal diameter of the protective sleeve 27 less a clearance.

FIG. 7f shows that a hole is formed in the second bone segment 34 by means of a drill 37 that extends through the drill sleeve 25, which drilled hole is coincident with the upper through-opening 6, arranged adjacent to the bearing portion 2, and continues on the other side in the second bone segment 34. Through this drilled hole formed in the second bone segment 34 and extending transversely with respect to the longitudinal extent of the bone plate 1, a bone screw can be inserted via the through-opening 6, and, in this way, the intramedullary portion 3 can be secured within the second bone segment 34.

FIG. 7g shows that, after the hole has been drilled into the second bone segment 34, the drill sleeve 25 is removed, after which, as can be seen in FIG. 7h, the depth of the drilled hole is measured using a flexible measuring instrument 38 in order to avoid inserting a bone screw that is too long and that passes through the second bone segment 34.

FIG. 7i shows how the last of four bone screws 40 is screwed into the second bone segment 34 by means of a screwdriver 39. Theoretically, only a single bone screw 40 is needed for fixation. It is better to fix the intramedullary portion 3 by means of at least two bone screws 40.

As will be seen from FIG. 7j, after all the bone screws 40 have been fitted, the insertion aid 18 is released and withdrawn from the bone plate 1 by suitable rotation of the fixing screw 30, after which the bearing portion 2 is fixed on the outside of the first bone segment 33, in this illustrative embodiment with the aid of three bone screws 41.

As will be seen from FIG. 7k, the bone segments 33, 34 that have been displaced laterally with respect to each other are fixed securely to each other by means of the bone plate 1, such that a twisting and/or shifting of the bone segments 33, 34 relative to each other is successfully prevented by the bone plate 1 with the bearing portion 2 and the intramedullary portion 3.

1. Foot surgery bone plate for osteosynthesis and/or fixation of foot-bone segments, comprising a bone plate having a plate-shaped bearing portion which has at least one through-opening for a bone screw extending from a top face of the bone plate to a bottom face of the bone plate, the bearing portion is designed for external fixation on a first bone segment, an intramedullary portion has at least one through-opening for the bone screw, extending from the top face of the bone plate to the bottom face of the bone plate, wherein intramedullary portion is designed for introduction into a second bone segment.

2. Bone plate according to claim 1, wherein the intramedullary portion has a free end which is pointed.

3. Bone plate according to claim 1, wherein the intramedullary portion includes several through-openings spaced apart from one another in a longitudinal direction of a intramedullary portion to receive in each case one bone screw.

4. Bone plate according to claim 1, wherein the bottom face of the bone plate has a concave curvature transverse to the bone plate longitudinal extent.

5. Bone plate according to claim 4, wherein material thickness of the bone plate, transverse to the longitudinal extent of the bone plate is less in the intramedullary portion than in the bearing portion.

6. Bone plate according to claim 5, wherein the bearing portion of the bone plate, in a transverse direction of the bone plate, is wider than the intramedullary portion.

7. Bone plate according to claim 1, wherein the bearing portion is provided with at least three through-openings, each for one bone screw, wherein a first through-opening is arranged on an imaginary continuation of a longitudinal center axis of the intramedullary portion, a second through-opening is offset axially from the first through-opening in a first transverse direction, and a third through-opening is axially spaced apart from the second through-opening and is offset from the first through-opening in a second transverse direction.

8. Bone plate according to claim 7, wherein the longitudinal center axes of at least two through-openings in the bearing portion extend at an angle to each other.

9. Bone plate according to claim 1, wherein the top face of the intramedullary portion has a central flat portion extending in a longitudinal direction, and, on each side of the flat portion, a flank portion also extends in the longitudinal direction, said flank portions being designed falling away toward the outside in the transverse direction.

10. System comprising a bone plate and an insertion aid for driving an intramedullary portion of the bone plate into a bone segment.

11. System according to claim 10, wherein means are provided for releasably securing a bearing portion of the bone plate on the insertion aid.

12. System according to claim 11, wherein the bearing portion is secured releasably on a bottom face of the insertion aid, and a strike face of the insertion aid extends perpendicular to the bottom face.

13. System according to claim 12, wherein the insertion aid has a drill jig.

14. System according to claim 13, wherein the drill jig extends at least approximately parallel to the bone plate secured on the insertion aid, and several through-holes spaced apart in a longitudinal direction of the drill jig are provided for passage of a drill, said through-holes being arranged in alignment with the through-openings of the intramedullary portion of the bone plate.

15. System according to claim 14, wherein at least one protective sleeve for receiving a drill sleeve, is provided which can be received in at least one of the through-holes, at least sectionally.

16. System according to claim 15, wherein at least one drill sleeve, for direct passage of a drill, is provided which can be received in the protective sleeve and/or in one of the through-holes, at least sectionally.
